(19) United States
(12) Patent Application Publication
(10) Pub. No.: US 2025/0235193 A1
(43) Pub. Date: Jul. 24, 2025

(54) CONTINUOUS-MOTION, LOW RADIODENSITY SURGICAL RETRACTOR FOR FACILITATING IMPROVED VISUALIZATION AND PRECISE RETRACTION OF A TISSUE OF A BODY

(71) Applicant: Alumina Medical LLC, San Francisco, CA (US)

(72) Inventors: Brendan WILLMS, Sacramento, CA (US); Hardik KAPOOR, Sacramento, CA (US); Patrick POPOVITS, San Francisco, CA (US)

(21) Appl. No.: 18/417,975

(22) Filed: Jan. 19, 2024

(51) Int. Cl. A61B 17/02; A61B 17/00

(52) U.S. Cl. CPC A61B 17/02; A61B 2017/00367; A61B 2017/00836; A61B 2017/0092; A61B 2017/00964; A61B 2017/00982

(57) ABSTRACT

A surgical retractor is provided with a plurality of gears, and first and second legs, each having a proximal end and a distal end. The first and second legs are constructed and arranged for the removable attachment of retractor blades thereto. An adjustment component is configured to be rotated in different directions. A plurality of gears provides a continuous rotational motion to the first and second legs when the adjustment component is rotated. The first and second legs move between the closed position and an unlimited number of open positions such that edges of a tissue of a body in contact with the retractor blades separate or are loosened from a recent separation. The retractor's low radiodensity will provide improved visibility of the surgical site and obviate the need to remove it from the surgical site for x-ray throughout the procedure.

### TECHNICAL FIELD

The present invention relates to surgical retractors, and more particularly, surgical retractors having a continuous motion and low X-ray radiodensity. Surgical retractors are surgical instruments that are used to hold an incision or wound open during a surgical procedure. They are used in various surgical specialties, including general surgery, spine surgery, orthopedics, ENT surgery, and neurosurgery.

### BACKGROUND

Surgical retractors are surgical instruments that are used to hold an incision or wound open during a surgical procedure. They are used in various surgical specialties, including general surgery, orthopedics, spine surgery, and neurosurgery.

X-rays are commonly used during surgical procedures to provide real-time imaging of the surgical site. Intraoperative X-ray imaging is particularly useful for procedures that involve bones or other hard tissues, such as orthopedic surgery or spine surgery. It allows the surgeon to see the position of bones and implants, check alignment, and ensure everything is in the correct place. During an intraoperative X-ray, the patient is positioned on the operating table and the X-ray machine is brought into the surgical suite. The surgeon or a radiologic technologist then takes the X-ray while the surgical team wears protective gear to minimize their exposure to radiation. The images produced are typically displayed on a monitor in the operating room, allowing the surgeon to see the anatomy in real-time.

Traditional surgical retractors, such as the widely used Black Belt, have been in use since the 1970s. Traditional retractors rely on a ratcheting mechanism that does not allow the surgeon or other medical professional to choose an optimal opening width. Instead, the ratcheting mechanism allows the retractor to “lock” at incremental positions spaced approximately 2 mm apart. In addition, to withdraw a traditional retractor, a medical professional must further extend the retractor before it can be loosened and the tissue released. In both situations, additional tissue is retracted, which may impact healing. Lastly, if the surgeon wants to decrease the amount of retraction during the surgery, the ratcheting mechanism makes it difficult to decrease the forces on the tissues by collapsing the device when released. These limitations make repositioning a traditional surgical retractor difficult.

The ratcheting mechanisms of traditional surgical retractors are uncovered and within the surgical field, meaning they could snag on someone's clothing or otherwise interfere due to their geometry.

Traditional surgical retractors are made from medical grade stainless steel, which is a radiopaque material, meaning it is visible under x-ray. X-rays are frequently used during surgery to find the specific surgical site and to determine the position of implants. Radiopaque retractors make it difficult to achieve this visibility. As a result, traditional surgical retractors must be removed from the surgical site before intraoperative x-ray imaging can be utilized. After the x-rays are taken, the retractor would need to be reinstalled before surgery continues, which significantly increases operating time which is always a patient safety risk.

### BRIEF DESCRIPTION OF THE DRAWINGS

Based on these and other needs in the surgical field, it is therefore desirable to provide a system, apparatus, and method, which solves at least some of the drawbacks associated with traditional surgical retractors.

In one aspect of the present disclosure, a surgical retractor is provided with a plurality of gears, and first and second legs, each having a proximal end and a distal end, the first and second legs are constructed and arranged for the removable attachment of retractor blades thereto, an adjustment component configured to be rotated in different directions, the plurality of gears in connection with said adjustment component for providing a continuous rotational motion to the first and second legs when the adjustment component is rotated, and the first and second legs configured to move between the closed position and a plurality of open positions such that edges of a tissue of a body in contact with the retractor blades separate or are loosened from a recent separation. The continuous rotational motion allows more precise adjustments as opposed to traditional ratcheting mechanisms. Additionally, the rotational motion allows the surgeon to close retractor slightly to decrease the pressure on the tissue preventing injury without losing retraction of the surgical site. Traditional ratcheting mechanisms collapse under the pressure of the tissues when attempting to achieve this.

### BRIEF DESCRIPTION OF THE DRAWINGS

The objects and advantages of the present disclosure will be apparent upon consideration of the following detailed description, taken in conjunction with the accompanying drawings, in which like reference characters refer to like parts throughout, and in which:

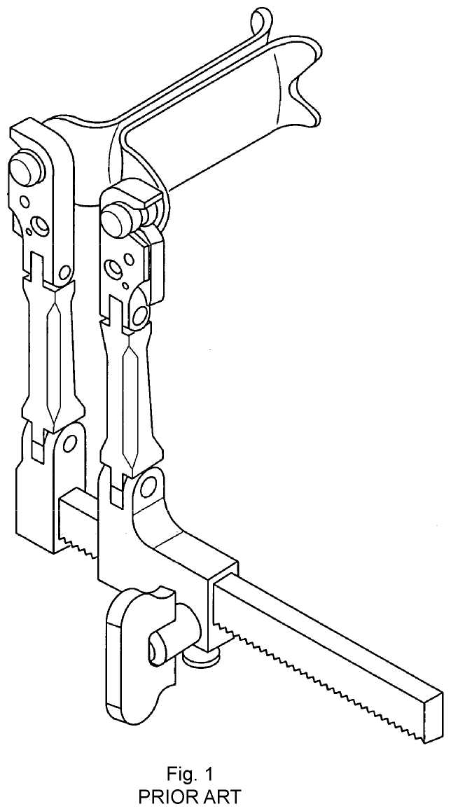

FIG. 1 shows a prior art surgical retractor with a ratcheting design.

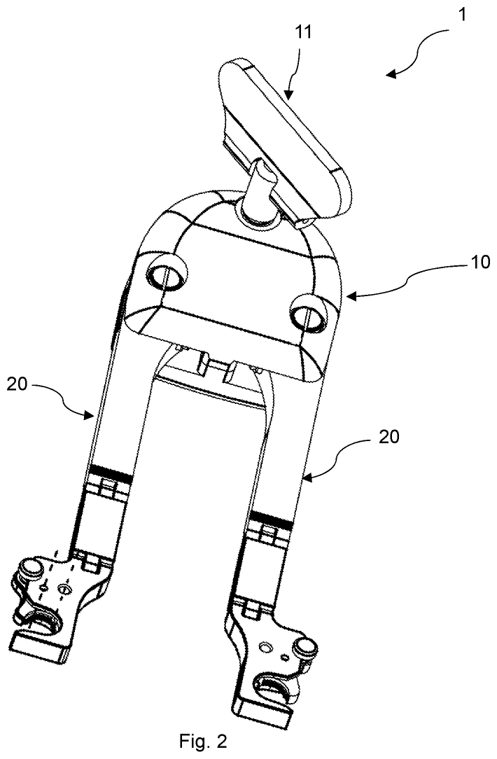

FIG. 2 shows an isometric view of an illustrative surgical retractor in accordance with the present disclosure.

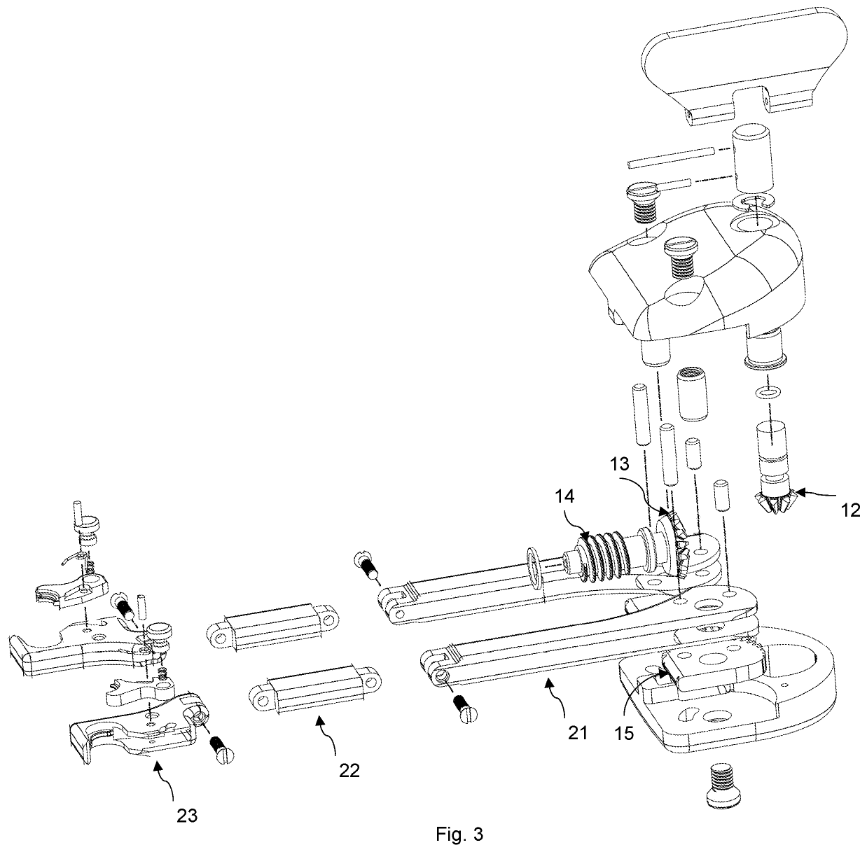

FIG. 3 shows an exploded view of an illustrative surgical retractor in accordance with the present disclosure.

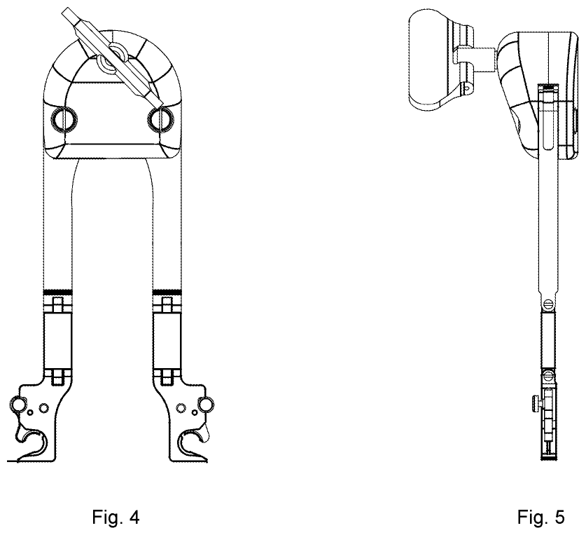

FIG. 4 shows a top view of an illustrative surgical retractor in accordance with the present disclosure.

FIG. 5 shows a side view of an illustrative surgical retractor in accordance with the present disclosure.

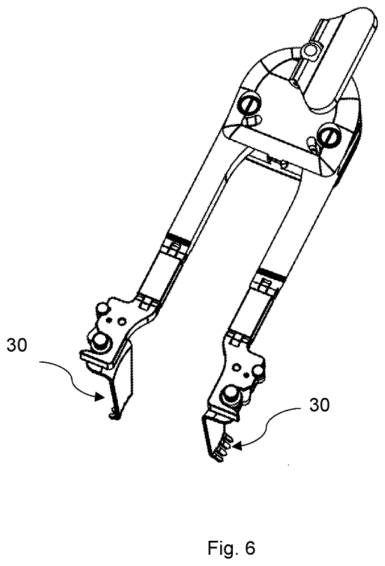

FIG. 6 shows an isometric view of an illustrative surgical retractor with retractor blades having fingers in accordance with the present disclosure.

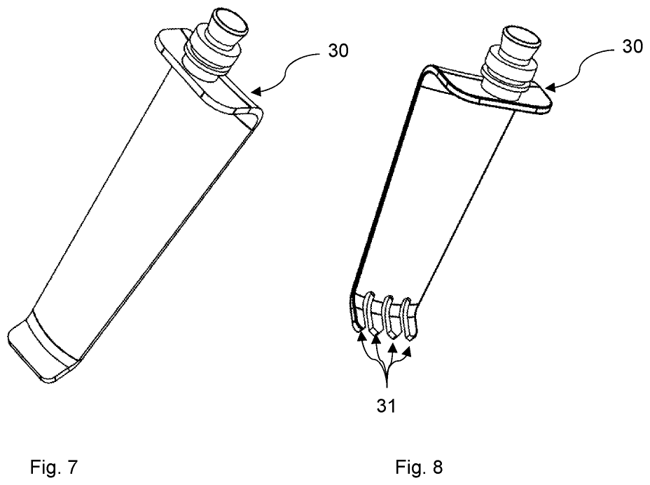

FIG. 7 shows an isometric view of an illustrative retractor blade without fingers.

FIG. 8 shows an isometric view of an illustrative retractor blade with fingers.

### DETAILED DESCRIPTION OF THE INVENTION

FIG. 1 shows a prior art surgical retractor with many of the disadvantages of a traditional surgical retractor discussed above. For example, the ratcheting mechanism is exposed, lends itself to only stepwise adjustment, and is made of stainless steel, making it obscure surgical details on x-ray imaging.

As shown in FIGS. 2-5, a surgical retractor 1 is provided as an embodiment in accordance with the present disclosure. The surgical retractor 1 has a housing 10, an adjustment component, such as a knob 11, and legs 20.

The housing 10 encloses a gear train designed to convert movement of the knob 11 into a separation of the legs 20. The housing 10 may enclosed the gear train, in part, to ensure the gear train does not come in contact with the patient during surgery. Since the housing and gear train would not appear in an intraoperative x-ray because they are outside the imaged area, this provides the added benefit of allowing the gear train to be constructed of any suitable material, such as stainless steel to name just one non-limiting example.

The knob 11 may rotate in a clockwise or an anticlockwise direction. Clockwise motion may cause the legs 20 to separate, while anticlockwise motion may cause the legs 20 to come closer together, or vice versa. The knob 11 may be configured to be presented to a user in an axis substantially orthogonal to both the longitudinal axis of the legs 20 and the plane upon which they spread to facilitate use of the knob during surgery. While a knob 11 is depicted, any number of angular or linear control mechanisms may be used as input to the surgical retractor.

The legs 20 extend from the housing 10 at their proximal end and may be substantially parallel or side-by-side in some configurations. The legs 20 are initially in a closed configuration (as shown in FIG. 1) and are configured to separate from one another. For example, the legs 20 may be configured to rotate such that an angle between them expands to at least 150 degrees, at least 157 degrees, at least 160 degrees, or at least 170 degrees.

Importantly, legs 20 are moved by the gear train in a continuous fashion. In prior art surgical retractors, the retractor legs are moved in stepwise increments, such as 2 millimeters. In contrast, the present surgical retractor 1 allows its legs 20 to be fine-tuned to, for example, 0.1-millimeter precision to name just one non-limiting level of precision. Such sub-millimeter precision allows greater control and accuracy during surgery. The precision can be modified by adjusting the gear train to reach desired results.

The legs 20 are configured to hold retractor blades 30 at their distal end, as shown in FIGS. 6-8. Retractor blades 30 may lock into place such that displacement of the retractor blades 30 relative to the legs 20 does not occur during use. Rotation of the blades is allowed, providing natural anatomic alignment with the retracted tissue. Locking mounts suitable for retractor blades 30 are well known in the art and include but are not limited to bayonet connections. In addition, retractor blades 30 are well known in the art and can be selected for size or other variables depending on the use scenario. For example, the retractor blades 30 may be in lengths from about 30-75 millimeters, and in width from about 10 to 25 millimeters, and may or may not have fingers 31 at their distal end. Retractor blades 30 may be tapered such that the width at the distal end is shorter than the width at the proximal end.

The legs 20 may be segmented. Such segmentation may allow for easier cleaning or replacement of parts. For example, as shown in FIG. 3, legs 20 have first segment 21, second segment 22, and third segment 23. First segment 21 may be directly connected to gear train at the proximal end of first segment 21. Second segment 22 is connected to the distal end of first segment 21 and the proximal end of third segment 23. Third segment 23 includes the mounting structure for retractor blades 30 at its distal end. The characteristics (e.g., length, width, etc.) and number of segments may be chosen to fit needs by a person having ordinary skill in the art.

As shown in FIG. 3, the housing 10 may include a gear train. In some embodiments, the gear train includes a bevel gear 12 connected to the knob 11. The bevel gear 12 may drive a pinion gear 13. The pinion gear 13 may be constructed on the same part as a worm gear 14. The worm gear 14 may drive two additional gears 15 which rotate in opposite directions during rotation of the worm gear 14. The rotation of these gears 15 causes the legs 20, each of which is operatively coupled to one of the gears 15, to separate.

By using a gear train instead of a ratcheting mechanism, the surgical retractor 10 improves upon the prior art. The level of separation of the legs 20 is continuously adjustable and not separated into discreet, segmented jumps. This allows a surgeon or other medical professional the ability to fine tune the positioning of the surgical retractor without the corresponding limits of a ratcheting device.

To install the surgical retractor 1, the retractor blades 30 are inserted into the incision or wound along an appropriate axis. The knob 11 is rotated in appropriate direction to cause the legs 20 to separate such that the incision or wound is opened. For example, the knob 11 may cause a rotation of the bevel gear 12, the pinion gear 13, and the worm gears 14, 15 such that the set of legs rotates about an axis such that edges of a tissue separate.

Unlike in the prior art, there is no need to take any steps to lock or otherwise immobilize the legs 20.

To withdraw the surgical retractor 1, the knob 11 is rotated in the opposite direction, causing the legs 20 to move closer together. For example, the knob 11 may cause a rotation of the bevel gear 12, the pinion gear 13, and the worm gears 14, 15 such that the set of legs rotates about an axis such that edges of a tissue are loosened from a recent separation. The surgical retractor 1 may then be withdrawn from the incision or wound. Unlike in the prior art, withdrawing the surgical retractor 1 does not require further separation of the legs 20, as the legs do not have to be momentarily further separated before the legs can be moved closer together, as is required in the prior art retractors once they are locked in a position.

The surgical retractor 1 may be constructed from a material designed to reduce the surgical retractor's 1 visibility on an x-ray image. For example, the medical retraction 1 may be constructed from aluminum (such as aluminum 6061), carbon fiber, or titanium materials, to name just a few non-limiting examples. As compared to materials used in prior art surgical retractors, such as medical grade stainless steel, aluminum 6061, Carbon fiber and titanium have low radiodensity. It may be possible that only a portion of the surgical retractor 1 be constructed from such a low visibility material to obtain the desired benefit. For example, the legs 20, retractor blades 30, and the constituent parts thereof, may be constructed from a radiolucent material with low x-ray opacity, while portions of the gear train may be made from medical grade stainless steel, since they do not generally appear on the x-ray image while the surgical retractor is used in surgery. Since the surgical retractor 1 is made from a less x-ray opaque material, it does not need to be removed from the surgical site when intraoperative or other x-ray imaging technology is used.

Thus, a surgical retractor has been provided. Persons skilled in the art will appreciate that the present invention can be practiced by other than the described examples, which are presented for purposes of illustration rather than of limitation.

What is claimed is:

1. A surgical retractor, comprising:
a plurality of gears;
a first leg comprising a first leg proximal end and a first leg distal end;
a second leg comprising a second leg proximal end and a second leg distal end;
the first and second legs constructed and arranged for the removable attachment of retractor blades thereto;
an adjustment component configured to be rotated in different directions;
the plurality of gears operably coupled between said adjustment component and said first and second legs for providing a continuous rotational motion to the first leg about the first leg proximal end and to the second leg about the second leg proximal end when the adjustment component is rotated; and
the first and second legs configured to move between the closed position and a plurality of open positions such that edges of a tissue of a body in contact with the retractor blades separated or are loosened from a recent separation, depending on the direction of rotation of the adjustment component.

2. The surgical retractor of claim 1, further comprising a housing enclosing the gears.

3. The surgical retractor of claim 2, wherein the plurality of gears is enclosed in the housing such that solids, liquids, and gases cannot enter the housing when the surgical retractor is in use.

4. The surgical retractor of claim 1, wherein the adjustment component comprises a knob.

5. The surgical retractor of claim 1, wherein the first and second legs are made from a material selected from a group consisting of: aluminum, aluminum 6061, carbon fiber, plastic, and composite materials.

6. The surgical retractor of claim 10, wherein the retractor blades are made from a material selected from the group consisting of: aluminum, aluminum 6061, carbon fiber, plastic, and composite materials.

7. The surgical retractor of claim 1, wherein the plurality of gears is made from stainless steel.

8. The surgical retractor of claim 1, wherein the plurality of gears includes each of a bevel gear, a worm gear, and a pinion gear.

9. The surgical retractor of claim 2, the adjustment component extending from an axis perpendicular to the axis extending from the housing to distal ends of the legs in the closed position, and a plane defined by the movement of the legs between the closed position and plurality of open positions.

10. The surgical retractor of claim 1, further comprising first and second retractor blades removably connected to each distal end of the respective first and second legs.

11. The surgical retractor of claim 10, further comprising a set of retractor blades, wherein the first and second retractor blades are selected from the set of retractor blades.

12. A surgical retractor configured to facilitate positioning a tissue of a body, the surgical retractor comprising:
an adjustment component;
a bevel gear controlled by the adjustment component;
a worm gear rotationally coupled with the bevel gear;
a housing, in which the bevel gear and worm gear are disposed;
first and second legs operatively coupled to the bevel gear and the worm gear;
first and second retractor blades removably attached to respective ones of the first and second legs; and
the first and second legs and the first and second retractor blades having low X-ray radiodensity;
wherein actuation of the adjustment component causes a rotation of the bevel gear and the worm gear such that the first and second legs rotate about an axis such that edges of a tissue separate or are loosened from a recent separation.

13. The surgical retractor of claim 12, wherein the adjustment component is a knob.

14. The surgical retractor of claim 12, wherein the axis is transverse to one or more rotational axes of the bevel and worm gear.
